(19) United States
(12) Patent Application Publication
(10) Pub. No.: US 2025/0169717 A1
(43) Pub. Date: May 29, 2025

(54) SYSTEM FOR DETERMINING A MOBILITY AGE SCORE

(71) Applicant: Palarum LLC, Lebanon, OH (US)

(72) Inventors: Patrick Baker, Lebanon, OH (US); Glenn Wolfe, Dayton, OH (US); Brian Riesenberg, Lebanon, OH (US); David Mann, Brooklyn, NY (US); Taylor Ellis, Cincinnati, OH (US); Chris Baker, Lebanon, OH (US)

(73) Assignee: Palarum LLC, Lebanon, OH (US)

(21) Appl. No.: 18/840,217

(22) PCT Filed: Mar. 1, 2023
(86) PCT No.: PCT/US2023/014235

§ 371 Date: Aug. 21, 2024

Related U.S. Application Data
(60) Provisional application No. 63/315,269, filed on Mar. 1, 2022.

(51) Int. Cl. A61B 5/11; A41D 1/00; A61B 5/00

(52) U.S. Cl. CPC A61B 5/112; A41D 1/002; A61B 5/6807; A61B 2562/0247

(57) ABSTRACT

A system for analyzing the mobility of an individual and from it determining a mobility age score. The mobility age score is a single age-based mobility number indicating a relative mobility with respect to other individuals in different age categories. The mobility score is optionally calculated based, on data obtained from multiple sensors such as pressure sensors, accelerometers and the like which may be worn by the user. This input may be processed and compared to data about the mobility of numerous other individuals obtained, over time in order to arrive at the mobility age score.

### SUMMARY

Disclosed is a system for analyzing the gait and/or mobility of an individual. In one aspect, the system determines a single age-based mobility number that may be clinically relevant to a physician or other medical professional, and optionally to the individual as well in determining mobility, and trends in mobility over time such as while moving through a rehabilitation process. The disclosed mobility score is optionally calculated based on data obtained from multiple sensors such as pressure sensors, accelerometers, angle sensors and the like which may be worn by the user. This input may be processed according to the disclosed algorithms and methods, and compared to data about the mobility of numerous other individuals obtained over time in order to arrive at the mobility age score. The score is thus preferably accurate and repeatable, and is optionally obtained automatically with little or no manual data entry necessary.

### DETAILED DESCRIPTION

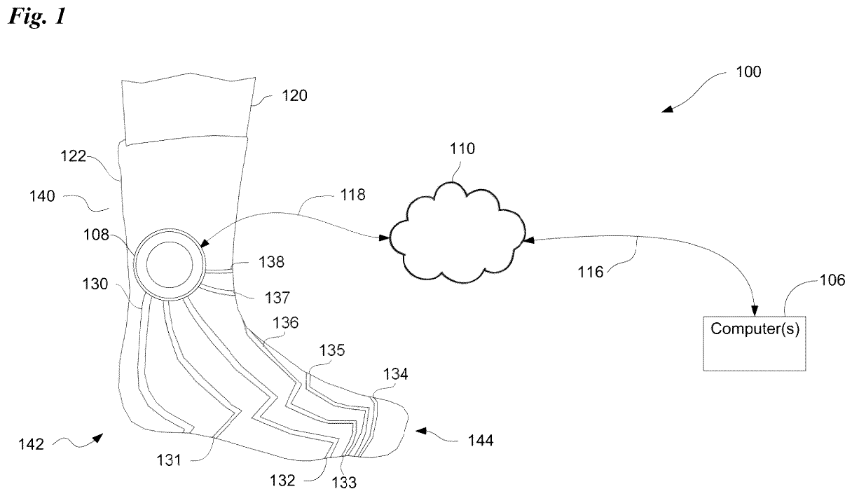

Illustrated at 100 in FIG. 1 is one example of components that may be included in a gait analysis system of the present disclosure. The system optionally includes a monitoring device 108 for detecting pressure placed on an extremity such as a foot or hand, movement, combinations of movements, positional changes, and other user activities or events that may provide insight into gait, mobility, and/or overall physical health.

Monitoring device 108 may be coupled to a user 120, for example, via a belt, an ankle bracelet, an armband, or as part of article of clothing such as a sock 122, shirt, gown, jacket, pants, and the like. For example, in FIG. 1, the monitoring device 108 is optionally mounted to a sock worn on a user's foot which may be configured to cover one or more of an ankle region 140, a heel region 142, and a toe region 144, or any combination thereof. In other examples, the monitoring device may be mounted to other articles of clothing on other locations of a user's body such as on a sleeve adjacent an arm or on a belt at the waist instead of, or in addition to, being worn in the ankle region.

Monitoring device 108 may communicate with and be responsive to one or more servers, databases, and/or other computing devices such as computers 106. These communications may be carried from monitoring device 108 to other devices using a communications links like communications links 116, and 118 that may also use a network 110. In one example, a computer 106 may be configured to discover what monitoring devices 108 are nearby using network 110, and may be configured to allow a caregiver using a computer 106 to select from which monitoring devices to monitor and receive gait analysis information.

Multiple circuits 130-138 may be included in sock 122 and electrically connected to terminals or contacts on monitoring device 108. Monitoring device 108 is preferably held firmly in place relative to the circuits in the sock so that specific circuits 130-138 may be electrically connected to corresponding terminals or contact pads of the monitoring device. Circuits 130-138 may also be electrically connected to one or more sensors responsive to corresponding sense parameters that may be included in the sock. These sensors are optionally configured to detect sense parameters that change according to various movements or other activities of the user. The sensors thus provide signals representative of the sense parameters (such as pressure, acceleration, movement, and others).

Circuits in the sock or other garment of the present disclosure (such as circuits 130-138) may, for example, include or be defined by one or more conductive threads woven into a portion of the fabric of the sock. In other examples, the disclosed circuits may be attached to the fabric by incorporating metallic traces into the garment fabric as a separate conductive layer between insulating layers in the fabric, or by conductive threads or traces into the sock fabric to create conductive regions operable as a circuit, or by adhering metallic traces to the fabric, or by any other suitable method.

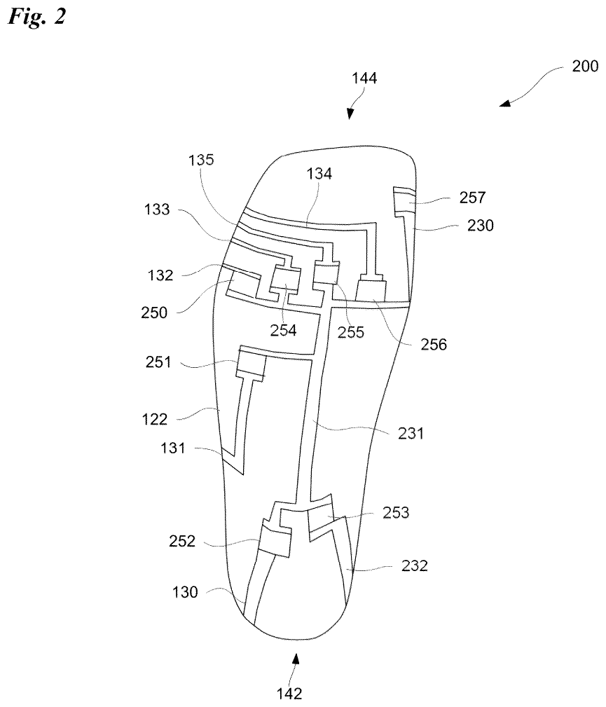

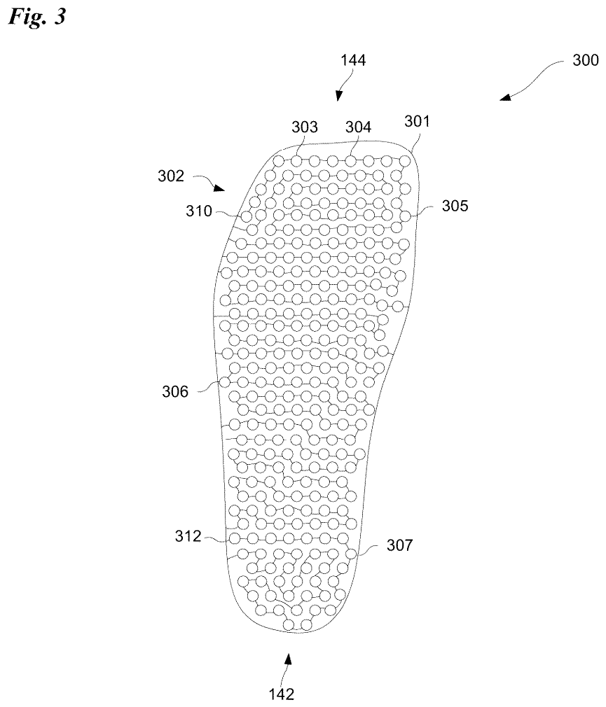

Examples of one or more sensors electrically connected to circuits like circuits 130-138 are illustrated in FIG. 2 at 200, and in FIG. 3 at 300. In FIG. 2, pressure sensors 250-257 are positioned on the sole or bottom of the sock 122. Any suitable positioning arrangement of sensors is envisioned. For example, sensors 250, 254, 255, and 256 are optionally arranged and configured to detect changes in pressure toward the front of the foot on the padded portion of the sole between the toes and the arch, sometimes referred to colloquially as “the ball of the foot”. Put another way, sensors 250, 254, 255, and 256 are optionally included in sock 122 at a location corresponding approximately with the forward or anterior portion of the metatarsal bones of the foot. This may be advantageous because pressures may tend to be highest in this region of the foot. Pressures may vary laterally across this region meaning that pressures at the location of sensors 254 and 255 and may be somewhat lower than in the areas of sensors 250, and 256. Such a positioning of sensors can be advantageous because when standing, overall pressure on this region of the foot corresponds with supporting a significant portion of the weight a user can place on the foot. Thus sensors 250, 254, 255, and 256 detecting changes in pressures in this location which can be helpful in determining that a user may be moving to a standing position.

In another example also illustrated in FIG. 2, sensors 252 and 253 are positioned toward the rear of the foot corresponding with a location on the sole adjacent to the heel region at 142. In this example sensor 252 may be positioned to detect pressure on the lateral hind foot region of the foot, while sensor 253 may be positioned to detect changes in pressure on the medial hind foot region of the foot. These regions correspond approximately with the talus and calcaneus bones of the foot. Positioning sensors in these general areas can be advantageous because as with the “ball of the foot”, overall pressure sensed at these locations correspond to supporting a significant portion of the weight placed on the foot by the user. In some examples, about half of the user's weight may be supported by the ball of the foot while most if not all of the remaining weight may be supported by the heel.

Other sensors may also be positioned on the sole of sock 122 which may be advantageous to further clarify whether a user is moving to a standing position, walking, carrying excessive weight, and the like. For example, sensor 251 may be placed adjacent the midfoot region approximately corresponding with the cuneiform bones of the foot. In another example, sensor 257 may be placed adjacent the toe region 144 of sock 122 to correspond with and measure pressure on the “big toe” or hallux of the foot. Any suitable arrangement of pressure sensors may be included to register pressures on other regions such as the individual toes, and the like.

In FIG. 3, numerous pressure sensors such as sensors 303-306 are positioned all around the sole or bottom of a sock 301 which is similar to sock 122. Any suitable positioning arrangement of sensors is envisioned. For example, a sensor array 302 may be arranged and configured to detect changes in pressure across substantially all of the length and breadth of the individual's foot. Individual sensors such as sensors 303-307 may be spread around the sock generally evenly distributed over the area, or they may be grouped together and localized at specific regions of interest such as the ball of the foot, the heal, the midsection, and so forth. Thus the greater number of separate sensors allows measurement of pressure at many locations corresponding approximately with the different regions of the foot. The increased number of sensors optionally provides additional granularity in data retrieved from the sensor array 302 thus allowing for more precise determinations of pressure distribution of the foot over time as the individual walks, runs, stands, etc.

In another aspect, changes or variations in lateral pressures that may vary across different regions of the foot may be more accurately determined. With the increased number of sensors 303-307, more precise pressure gradients may be measured showing the changes in pressure across the surface area of the sock 301. As with the examples shown in FIG. 2, and elsewhere, the sensor array 302 is optionally configured to detect pressure on the toes at 144, on the ball of the foot, on the lateral hind foot region of the foot 142, or the medial hind foot region of the foot, regions which correspond approximately with the talus and calcaneus bones of the foot. Sensors may be placed adjacent the midfoot region approximately corresponding with the cuneiform bones of the foot, or in the “big toe” or hallux region of the foot. Any suitable arrangement of pressure sensors may be included to register pressures on other regions such as the individual toes, and the like.

Like FIG. 2, circuits may be included in sock 301 to electrically connect individual, or multiple sensors in the array 302 to a monitoring device like monitoring device 108. For example, circuit sensors 303 and 304 may be electrically connected to circuit 310 while sensor 307 may be electrically connected to circuit 312. Circuits 310 and 312 (and others illustrated in sock 301) may be electrically connected to power, ground, and/or data input circuits of a monitoring device useful for detecting pressure gradients across sock 301. In another aspect, circuits shown in FIGS. 2 and 3 may include any suitable number of separate wires or cables for carrying pressure data from the sensors to the monitoring device.

The sensors shown in FIGS. 2 and 3 may be of any suitable type or construction. For example, sensors 250-257 and/or sensors 303-307 may include piezoresistive fibers, which is to say, fibers which change resistance as the pressure exerted on them changes. In one preferred embodiment, these resistive fibers may be woven together with the threads of the fabric in the general area of locations of the sensors shown in FIGS. 2 and 3. Thus the sensors may alternatively be characterized as regions of the garment fabric with increased sensitivity to changes in pressure resulting from the addition of resistive fibers. In other examples, a pressure sensor maybe be inserted into the garment at 250-257, or in any of the locations shown in the sensor array 302, such as by removing a portion of the fabric and replacing it with a sensor, or with other fabric that includes the resistive fibers. A pressure sensor may also be inserted into the fabric and maintained between the inside and outside surfaces. In another example, the pressure sensors may be attached to the fabric such as by an adhesive or by other means. Thus pressure sensors may be included with the fabric by any suitable technique, but preferably without substantially increasing the thickness of the garment fabric, and/or without adding heavy or bulky devices that are unpleasant for the user. Preferably, the sensors are mounted in a way that is unobtrusive and comfortable for the user.

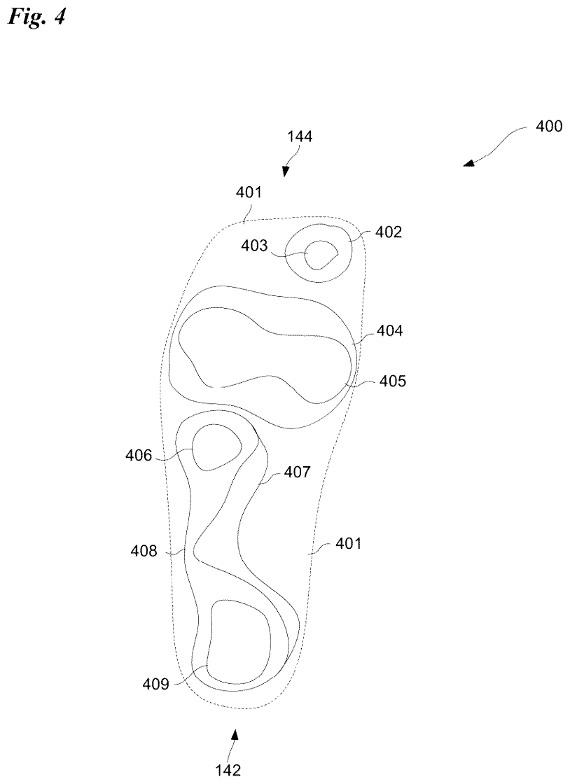

FIG. 4 illustrates at 400 examples of pressure gradients that may be obtained using the pressure sensors of the present disclosure. An area of average or baseline low pressure 401 may generally correspond to areas of the sock where minimal pressure is present. An area of increased pressure in the area of the big toe may be determined at 402, with further increases in pressure shown at 403. Similarly, an area of increased pressure around the middle portion of the foot, such as at the ball region may appear at 404, with further increases in pressure at 405. Likewise, region 407 represents an area of somewhat elevated pressure, region 408 being an area of further elevated pressure, and the areas of still greater pressure indicated at 406 and 409.

In another aspect, these pressure gradients obtained from the disclosed sensors may indicate different areas of the sock that are experiencing approximately the same levels of pressure. For example the region defined by 404 may be experiencing about the same pressure throughout this region. In another aspect, the regions illustrated at 400 may vary in pressure within a specified range, and thus may be represented as a single region. In another aspect, pressure gradients may also modify over time as pressure on the corresponding regions changes based on movement of the individual user. The changes in these pressure gradients over time, or while performing particular predetermined tasks may be used in the algorithms of the present disclosure to determine a user's mobility score or mobility age.

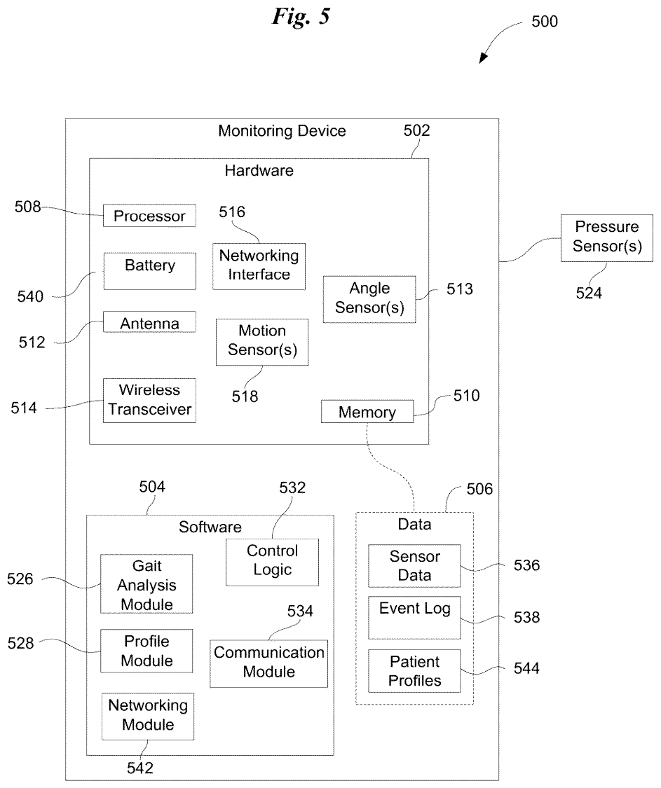

Additional software, hardware, and data aspects of a monitoring device of the present disclosure (like monitoring device 108) are further illustrated in FIG. 5. The monitoring device of the present disclosure may include any suitable electronic components and/or software for monitoring user activity. FIG. 5 illustrates at 500 one example of hardware and software that may be included in monitoring device. A monitoring device may generally include hardware 502, software 504, and may optionally include a local data store 506.

Hardware 502 may include a processor 508 which may be programmed or configured to perform tasks discussed herein related to monitoring a user's activity, analyzing the user's gait, and/or determining an overall age score based on this input. Processor 508 may be coupled to other aspects of hardware 502 such as sensors, memory, and the like to perform these tasks. Memory 502 may be included for storing operating values or parameters which may include intermediate calculated values, or final calculated values, logical or computational instructions for processor 508, hardware control parameters, and the like. Memory 502 may also store user monitoring information such as user related events in an event log 538, sensor data 536 obtained from sensors coupled to the monitoring device, and/or user profiles 544 for controlling how data about user activity is collected and analyzed. Memory 502 may be either a permanent or “static” memory, or a temporary or “dynamic” memory, or any combination thereof.

An antenna 512 may be included to facilitate wireless communications over a communication link like communication link 118. A networking interface 516 may be included to establish and maintain communications with other devices via a network such as network 110. Wireless transceiver 514 may be included and may use antenna 512 or other suitable hardware 502 to transmit and receive information between control module 500 and other devices in the user monitoring system such as servers, data stores, and/or computers like computers 106.

Control module 500 may include one or more sensors such as one or more motion sensors 518 configured to detect a user's movements. Motion sensors 518 may include any suitable device or devices responsive to the movement of the user and may include, for example, one or more accelerometers to detect movement in multiple axes relative to gravity, and/or one or more gyroscopic sensors for detecting changes in angular momentum, direction of travel, and/or an angle of elevation. Motion sensor 518 may be used to detect when a user is walking, sitting, running, or making other movements, when a user changes position to stand, sit, lay down and the like.

The control module may also include one or more angle sensor(s) 513 which may be arranged and configured to detect changes in angles. For example, the angle sensors 513 may be useful for determining changes in angles of one body part relative to another, or relative to gravity. In one example, the angle sensors include a gyroscope configured to be mounted adjacent an ankle, a knee, elbow, or other area of a human body. These additional angle inputs provided by sensors 513 may be useful, for example, in determining when user is walking in straight line, moving up hill or downhill, or turning.

One or more pressure sensors 524 may also be included, and may be useful for detecting changes in the distribution of pressure on a user's body. For example, pressure sensors 524 may detect a pressure gradient across a user's foot based on their walking or running gait, and this information, and how it changes over time, may be used to calculate their age score.

Any of the sensors used by control module 500 such as sensors 518, 524, and others, may be mounted inside or outside a housing containing some or all of the other hardware and software components. For example, sensors may be mounted outside a container or housing and may communicate with hardware and software inside the housing by any suitable communications link. For example, pressure sensor 524 may be woven into a user's clothing such as into a sock or gown, and may communicate with components of software 506 and hardware 502 mounted inside the housing via a wired or wireless communications link. This communications link may be maintained as electromagnetic signals traveling over wire leads, or through the air as radio waves using any suitable wireless communication technology.

These hardware aspects of control module 500 may be configured to operate according to instructions included in software 504, keeping in mind that the software 504 may be installed or embedded partially or completely within hardware 502. These operating instructions may be logically or conceptually arranged as modules for controlling different functional aspects of the monitoring device. Functional aspects generally include obtaining, storing, and processing data from multiple sensors, detecting user movements and other activity, determining when to send alert notices to other parts of the system, retrieving or updating user profile information, and/or sending sensor data to a central archive to improve the performance of monitoring devices throughout the system.

Profile module 528 may be configured to accept or modify or otherwise maintain a user profile 544. User profile 544 may include multiple parameters detailing information about the user, the user's treatment plan, and other information useful to control module 500 and the rest of user monitoring system 100. A user profile may include any information about the user useful for determining a mobility age score. Such information may include detailed user measurements such as medical condition, height, weight, body composition, gait analysis data obtained in the past, and previous mobility age scoring data. It may also include demographic information such as sex, race, and the like.

Parameters, or parameter ranges may be specified in any suitable format such as numbers, letters, binary data, and the like. For example parameters may be organized to correspond with input values required by one or more rules in alarm module 526. In another example, user parameters may be configured to correspond with output ranges of specific sensors or combination of sensors used by control module 500. The user parameters may be thought of as predetermined threshold values that may be compared to sensor or other data according to a rule. These predetermined threshold values may be specific values or ranges of values, with or without accompanying tolerances. Such values may be numerical, textual, or any combination thereof.

Control logic 532 may be included to organize the operations of software 504 and/or hardware 502. Control logic 532 may be configured to initialize the activity of control module 500 such as going through a basic startup and testing procedure, running through algorithms or subroutines to locate and communicate with remote servers or databases, or other computers like computer 106, and/or other devices in the user monitoring system. Control logic 532 may be thought of as a “controller” that controls the operation of user control module 500. The hardware and software of the monitoring device may operate to begin one or more control loops periodically or continuously obtaining sensor data from one or more sensors in the monitoring device such as a motion sensor 518, or optionally one or more pressure sensors 524 which may be mounted elsewhere. These pressure sensors may be in any suitable location or mounted in any suitable way to obtain relevant pressure information useful for gait analysis. These may include, but are not limited to, the pressure sensors illustrated in FIGS. 3 and 4 which may be mounted or incorporated into a sock or other garment.

A communication module 534 may be included as well. Communication module 534 may be configured to open and maintain communication links to various other parts of the user monitoring system such remote servers or databases, and others. Communication module 534 may be configured to implement any suitable digital, analog, or other communication scheme using any suitable networking, or control protocol. Communication module 534 may engage or use networking module 542 to open, maintain and manage communication links with other aspects of the user monitoring system via network.

In one example, communications module 534 may be configured to automatically establish communication link 118 with network 110. User control module 500 may be configured to operate according to the IEEE 802.15 wireless networking standard (sometimes referred to as a “Bluetooth” or Wireless Personal Area Network or “WPAN”). In this example, communications module 534 may automatically interact with routers, switches, network repeaters or network endpoints, and the like to establish a communications link 118, and/or 112 so that event updates may be automatically configured to pass to a remote server where they may be processed and distributed. Communications module 534 may be implemented to use any combination of Generic Access Profile (GAP), Generic Attribute Profile (GATT), and/or Internet Protocol Support Profile (IPSP) protocols to acquire and maintain communications with remote servers, databases or computers.

Software 504 may include a gait analysis module 526 which may be implemented as software that programs or configures the processor, or other hardware in the system, to obtain and analyze movement data that is the result of user activity, and sends the results of that analysis and/or raw data to other parts of disclosed system. In one example, the gait analysis module 526 may implement an algorithm for determining a mobility score that includes a mobility age weighted average that is based on or incorporates a regression of gait speed, gait cadence, stride duration and stride length. In another aspect, the gait speed, cadence, and stride length are optionally normalized to height.

In another aspect of the gait analysis module 526, determining the users mobility age score may include calculations such as an integration of sagittal acceleration data (optionally excluding or subtracting gravity) to determine step length and time. The integration and timing may optionally begin at each toe-off event and may optionally end at heel strike of the same foot. In another aspect, integrations of step length may be used to determine average stride length.

These calculations may involve real time data processing of data obtained from the disclosed motion, angle, pressure, or other sensors that is optionally performed by the gait analysis module 526. These calculations may include determining a step has occurred based on data obtained from the disclosed sensors that falls within the limits of an average step. In another aspect of the gait analysis module, an average step may be determined using real time averaging of data provided by the sensors of the control module. An average step may optionally be calculated by the gait analysis module 526 by performing various statistical or numerical analysis on data such as pressure, motion, angle, or other gradients obtained via the disclosed sensors. This numerical processing and analysis may include calculating the mean, mode, median, standard deviation, or other aspects of the data, or on calculated values derived from the data.

In another aspect, the real time data processing performed according to the instructions of the gait analysis module 526 may determine when a count of typical steps reaches a minimum target threshold before executing the gait analysis algorithm of the present disclosure. In another aspect, the real time processing may determine when a user is starting to walk, stopping, or turning. According to the present disclosure, a manual measurement and entry of test distance is preferably eliminated in this instance, and possibly in others.

In another aspect, the gait analysis module 526 may be configured to determine when a user has begun taking steps, or calculate the mobility score by optionally excluding the initial starting steps, the final stopping steps, and steps taken while turning around. These start, stop, and turning steps may be evaluated differently with different parameters, or excluded from the calculations to reduce or eliminate the impact of these values on the overall mobility determination to preferably improve the overall accuracy of the disclosed algorithm. In another aspect, the gait analysis module optionally combines step times with stride length to determine averages for gait speed and cadence. In one example, gait speed may be equal to distance divided by time, stride length may be equal to the product of 2 multiplied by the distance, and then divided by the number of steps, and cadence may be equal to the number of steps divided by the time. In another aspect, these values may optionally be normalized according to height.

In another aspect, the gait analysis module 526 may determine an estimated step distances using experimental data collected from actual test subject that may be useful for indicating the relationship between step length and swing time where swing time is defined as the time a foot is off the ground during each step. These values are optionally normalized by height/inseam length and may be useful to augment or validate swing time measurements collected via the disclosed pressure, motion, angle, or other sensors, or any combination thereof.

In another aspect, the gait analysis module 526 may include, or optionally use third party software to integrate data from a shoe or ankle located monitoring device of the present disclosure. The third party software may be implemented as part of gait analysis module 526, or it may be executed as needed by the gait analysis module to support the disclosed computations. For example, the third party software may be compiled with code in the gait analysis module, or it may be linked with code in the gait analysis module so that the it may be called as needed. In another aspect, the third party software may be implemented as a web service, remote procedure call, or other similar service that may be called by the gait analysis module 526.

In another aspect, the third part software may implement an algorithm based on Zero Velocity Updates (ZUPT) which may provide the gait analysis module 526 with an effective way to suppress the growth of errors in inertial calculations made by devices located at or near a user's foot. This approach optionally integrates the data obtained from the disclosed sensors to determine the direction and length of individual steps. The third party software may optionally be configured to prevent cumulative integration errors, such as by resetting each time the motion determined by the sensor input meets one or more zero velocity criteria. In another aspect, mounting the motion sensors in the monitoring device adjacent or on an ankle region would exhibit different zero velocity characteristic from a monitoring device mounted adjacent to or on the top of a shoe, whereas the pressure sensor readings from the bottom of the socks would be the same regardless of where the monitoring device is mounted. Thus the third party software may provide an optional algorithm to, among other things, determine zero velocity conditions.

For example, the gait analysis module 526 using a zero velocity update algorithm (whether supplied by a third party or not) may be configured to discard steps having lengths that are outside predetermined acceptance criteria. These may include start, stop, and turning steps which may be evaluated differently with different parameters, or excluded from the calculations to preferably improve the overall accuracy of the mobility score. Steps may be counted by noting changes in pressure observed by the pressure sensors of the present disclosure. The distance walked by the user may be determined by the gait analysis module by, for example, summing over multiple steps, where each step is optionally determined according to the ZUPT algorithm, and/or other algorithms of the software.

In another aspect, the stride length may be calculated by adding the step length for the left foot to the step length of the right foot. The distance walked may be optionally determined by summing together the individual step lengths. The time to complete the steps may be calculated by summing together the stride time for the left foot, and the stride time for the right foot. Gait speed may be calculated as the distance walked divided by the time taken to complete the steps. Cadence may be determined by dividing the steps taken by the time to complete the steps.

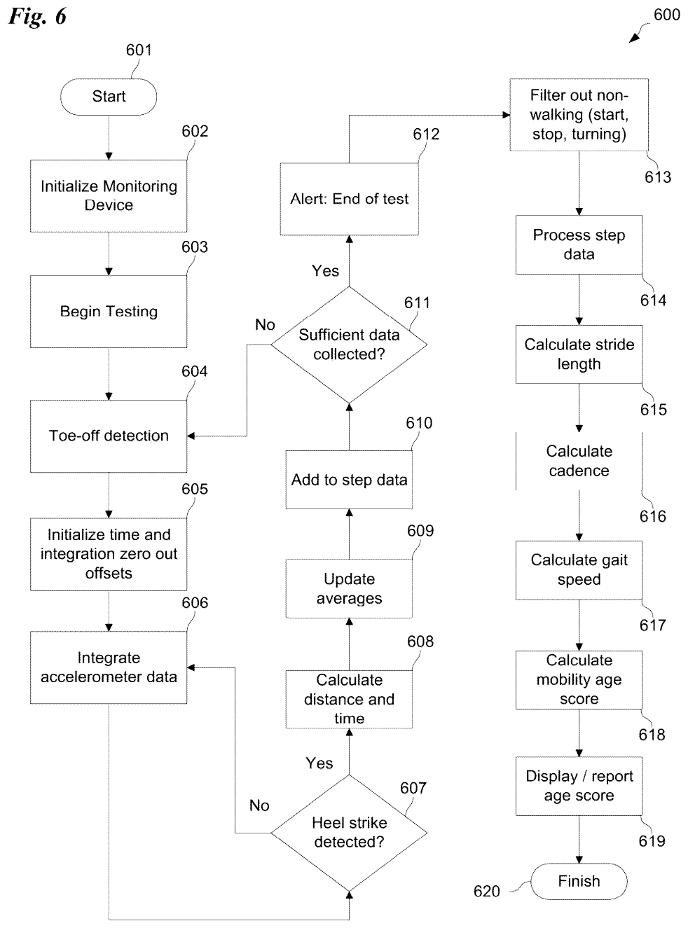

One example of a method of operating the disclosed system for determining a mobility age score is illustrated in FIG. 6 at 600. Starting at 601, the monitoring device may be initialized at 602 where one or more startup and/or calibration routines may be executed in order to prepare the monitoring devices to being reading from sensors, calculating results, establishing communication links with remote computing devices, and the like. In another aspect, initializing the monitoring device may include determining a gravity vector according to input from one or more sensors such as a motion sensor, angle sensor, and the like. In another aspect, the system may be operable to determine the orientation of the Sagittal plane as part of the initialization stage at 602.

Testing may begin at 603 and may be initiated by any suitable means. For example, testing may be initiated by input accepted from the user. This input may be captured by any suitable input device, such as via a touch screen of a smart phone executing an application. In another example, the monitoring device may include a user interface with buttons, one of which may be useful for initiating a test. In another example, a remote computing device in communication with the monitoring device may send a command to the monitoring device to initiate a test remotely. In yet another example, the remote monitoring device may be optionally configured to automatically initiate a test when certain criteria are met. Such criteria may include, but are not limited to; when the monitoring device determines that a user wearing the monitoring device has moved from a sitting to a standing position; when the monitoring device determines that a user is engaging in a particular activity such as after working out, during a long walk, or after prolonged periods of inactivity; when a predetermined period of time has passed since the last test was executed such as an hour, or four hours, or ten minutes, or any other suitable period of time; when another separate system such an automated healthcare management system that is in communication with the disclosed gait analysis system determines that a test should be run so as to monitor or report user activity; or when a predetermined time of day is reached, such as 3 pm, 9 am, and the like. Any suitable criteria for activation may be employed alone or in combination with input from a user, a clinician, another system, or another person.

At 604, the monitoring unit is optionally configured and arranged to detect a toe-off event, such as when a user picks up a foot. This event may be determined by the disclosed pressure sensors, such as might be mounted in a sock, shoe, or other garment adjacent a user's foot. A toe-off event is optionally determined when pressure gradients detected by the pressure sensors of the present disclosure match a predetermined profile. As pressures change across the user's foot, for example, the monitoring device may be configured to determine whether these pressure changes indicated the user has begun walking. In another aspect, pressure changes may be considered in combination with acceleration, velocity, angle relative to gravity, or other inputs that may be provided by the monitoring device. These additional inputs may provide a broad collection of time varying data inputs from which a profile of current activity may be obtained and compared with predetermined profiles.

The predetermined profiles may include corresponding time-varying data sets defining changes in pressure, acceleration, velocity, angles of inclination, etc. over time. These data sets for the predetermined profiles may be generated or obtained according to test data of individuals of different ages, body sizes and weights, physical capabilities, or according to software models of human behavior, and the like. These profiles may be stored in a memory of the monitoring device as a look-up table, or database, or in any suitable organization which allows for the monitoring device to rapidly compare time varying input defining user activity obtained from pressure, acceleration, or other sensors of the present disclosure.

At 605, the monitoring device may be configured to reset internal variables such as an initial time for the beginning of the test, and any temporary data values that may be left over from previous tests, if present. At 606, the system may be operable to integrate accelerometer data and to determine at 607 if a heal strike is detected. If not, the system may revert to integrating accelerometer data at 606. If so, a time and distance calculation may be performed at 608, and average for the various values calculated by the system may be updated at 609 according. These values may be added to the step data for the current test at 610.

If sufficient data is collected at 611, an alert may be generated that is observable to the user, a clinician, or other individual indicating that the test is completed at 612. This alert may be provided in any suitable form such as via an audible alarm generated by an audio output device, by a flashing lamp, by a phone call, text message, or via a notification displayed on screen or other output device. These output devices, or any combination thereof, may be mounted to the monitoring device, or to another device such as a remote computing device. If sufficient data has yet to be collected, the method reverts to detecting a toe-off condition at 604.

At 613, the disclosed gait analysis system optionally filters out non-walking steps from the data collected thus far. Such non-walking steps may include, but are not limited to, starting steps, stopping steps, or steps involved in turning around or changing direction. Additional processing may occur at 614, and calculations of stride length (615), cadence (616), and gait speed (617) may also be performed. These calculations are illustrated as a linear process, however, they may be undertaking in parallel at about the same time, or in a combination of linear and parallel.

At 618, the result mobility age score is determined and displayed for the user or clinician at 619. This final score may be displayed using an output device such as a screen, or it may be reported by any suitable output device including text messaging, printouts, and the like.

### Glossary of Definitions and Alternatives

While the invention is illustrated in the drawings and described herein, this disclosure is to be considered as illustrative and not restrictive in character. The present disclosure is exemplary in nature and all changes, equivalents, and modifications that come within the spirit of the invention are included. The detailed description is included herein to discuss aspects of the examples illustrated in the drawings for the purpose of promoting an understanding of the principles of the invention. No limitation of the scope of the invention is thereby intended. Any alterations and further modifications in the described examples, and any further applications of the principles described herein are contemplated as would normally occur to one skilled in the art to which the invention relates. Some examples are disclosed in detail, however some features that may not be relevant may have been left out for the sake of clarity.

Where there are references to publications, patents, and patent applications cited herein, they are understood to be incorporated by reference as if each individual publication, patent, or patent application were specifically and individually indicated to be incorporated by reference and set forth in its entirety herein.

Singular forms “a”, “an”, “the”, and the like include plural referents unless expressly discussed otherwise. As an illustration, references to “a device” or “the device” include one or more of such devices and equivalents thereof.

Directional terms, such as “up”, “down”, “top” “bottom”, “fore”, “aft”, “lateral”, “longitudinal”, “radial”, “circumferential”, etc., are used herein solely for the convenience of the reader in order to aid in the reader's understanding of the illustrated examples. The use of these directional terms does not in any manner limit the described, illustrated, and/or claimed features to a specific direction and/or orientation.

Multiple related items illustrated in the drawings with the same part number which are differentiated by a letter for separate individual instances, may be referred to generally by a distinguishable portion of the full name, and/or by the number alone. For example, if multiple “laterally extending elements” 90A, 90B, 90C, and 90D are illustrated in the drawings, the disclosure may refer to these as “laterally extending elements 90A-90D,” or as “laterally extending elements 90.” or by a distinguishable portion of the full name such as “elements 90”.

The language used in the disclosure are presumed to have only their plain and ordinary meaning, except as explicitly defined below. The words used in the definitions included herein are to only have their plain and ordinary meaning. Such plain and ordinary meaning is inclusive of all consistent dictionary definitions from the most recently published Webster's and Random House dictionaries. As used herein, the following definitions apply to the following terms or to common variations thereof (e.g., singular/plural forms, past/present tenses, etc.):

“Antenna” or “Antenna system” generally refers to an electrical device, or series of devices, in any suitable configuration, that converts electric power into electromagnetic radiation. Such radiation may be either vertically, horizontally, or circularly polarized at any frequency along the electromagnetic spectrum. Antennas transmitting with circular polarity may have either right-handed or left-handed polarization.

In the case of radio waves, an antenna may transmit at frequencies ranging along electromagnetic spectrum from extremely low frequency (ELF) to extremely high frequency (EHF). An antenna or antenna system designed to transmit radio waves may comprise an arrangement of metallic conductors (elements), electrically connected (often through a transmission line) to a receiver or transmitter. An oscillating current of electrons forced through the antenna by a transmitter can create an oscillating magnetic field around the antenna elements, while the charge of the electrons also creates an oscillating electric field along the elements. These time-varying fields radiate away from the antenna into space as a moving transverse electromagnetic field wave. Conversely, during reception, the oscillating electric and magnetic fields of an incoming electromagnetic wave exert force on the electrons in the antenna elements, causing them to move back and forth, creating oscillating currents in the antenna. These currents can then be detected by receivers and processed to retrieve digital or analog signals or data.

Antennas can be designed to transmit and receive radio waves substantially equally in all horizontal directions (omnidirectional antennas), or preferentially in a particular direction (directional or high gain antennas). In the latter case, an antenna may also include additional elements or surfaces which may or may not have any physical electrical connection to the transmitter or receiver. For example, parasitic elements, parabolic reflectors or horns, and other such non-energized elements serve to direct the radio waves into a beam or other desired radiation pattern. Thus antennas may be configured to exhibit increased or decreased directionality or “gain” by the placement of these various surfaces or elements. High gain antennas can be configured to direct a substantially large portion of the radiated electromagnetic energy in a given direction that may be vertical horizontal or any combination thereof.

Antennas may also be configured to radiate electromagnetic energy within a specific range of vertical angles (i.e. “takeoff angles) relative to the earth in order to focus electromagnetic energy toward an upper layer of the atmosphere such as the ionosphere. By directing electromagnetic energy toward the upper atmosphere at a specific angle, specific skip distances may be achieved at particular times of day by transmitting electromagnetic energy at particular frequencies.

Other examples of antennas include emitters and sensors that convert electrical energy into pulses of electromagnetic energy in the visible or invisible light portion of the electromagnetic spectrum. Examples include light emitting diodes, lasers, and the like that are configured to generate electromagnetic energy at frequencies ranging along the electromagnetic spectrum from far infrared to extreme ultraviolet.

“Battery” generally refers to an electrical energy storage device or storage system including multiple energy storage devices. A battery may include one or more separate electrochemical cells, each converting stored chemical energy into electrical energy by a chemical reaction to generate an electromotive force (or “EMF” measured in Volts). An individual battery cell may have a positive terminal (cathode) with a higher electrical potential, and a negative terminal (anode) that is at a lower electrical potential than the cathode. Any suitable electrochemical cell may be used that employ any suitable chemical process, including galvanic cells, electrolytic cells, fuel cells, flow cells and voltaic piles. When a battery is connected to an external circuit, electrolytes are able to move as ions within the battery, allowing the chemical reactions to be completed at the separate terminals thus delivering energy to the external circuit.

A battery may be a “primary” battery that can produce current immediately upon assembly. Examples of this type include alkaline batteries, nickel oxyhydroxide, lithium-copper, lithium-manganese, lithium-iron, lithium-carbon, lithium-thionyl chloride, mercury oxide, magnesium, zinc-air, zinc-chloride, or zinc-carbon batteries. Such batteries are often referred to as “disposable” insofar as they are generally not rechargeable and are discarded or recycled after discharge.

A battery may also be a “secondary” or “rechargeable” battery that can produce little or no current until charged. Examples of this type include lead-acid batteries, valve regulated lead-acid batteries, sealed gel-cell batteries, and various “dry cell” batteries such as nickel-cadmium (NiCd), nickel-zinc (NiZn), nickel metal hydride (NiMH), and lithium-ion (Li-ion) batteries.

“Communication Link” generally refers to a connection between two or more communicating entities and may or may not include a communications channel between the communicating entities. The communication between the communicating entities may occur by any suitable means. For example the connection may be implemented as an actual physical link, an electrical link, an electromagnetic link, a logical link, or any other suitable linkage facilitating communication.

In the case of an actual physical link, communication may occur by multiple components in the communication link configured to respond to one another by physical movement of one element in relation to another. In the case of an electrical link, the communication link may be composed of multiple electrical conductors electrically connected to form the communication link.

In the case of an electromagnetic link, the connection may be implemented by sending or receiving electromagnetic energy at any suitable frequency, thus allowing communications to pass as electromagnetic waves. These electromagnetic waves may or may not pass through a physical medium such as an optical fiber, or through free space, or any combination thereof. Electromagnetic waves may be passed at any suitable frequency including any frequency in the electromagnetic spectrum.

A communication link may include any suitable combination of hardware which may include software components as well. Such hardware may include routers, switches, networking endpoints, repeaters, signal strength enters, hubs, and the like.

In the case of a logical link, the communication link may be a conceptual linkage between the sender and recipient such as a transmission station in the receiving station. Logical link may include any combination of physical, electrical, electromagnetic, or other types of communication links.

“Communication node” generally refers to a physical or logical connection point, redistribution point or endpoint along a communication link. A physical network node is generally referred to as an active electronic device attached or coupled to a communication link, either physically, logically, or electromagnetically. A physical node is capable of sending, receiving, or forwarding information over a communication link. A communication node may or may not include a computer, processor, transmitter, receiver, repeater, and/or transmission lines, or any combination thereof.

“Computer” generally refers to any computing device configured to compute a result from any number of input values or variables. A computer may include a processor for performing calculations to process input or output. A computer may include a memory for storing values to be processed by the processor, or for storing the results of previous processing.

A computer may also be configured to accept input and output from a wide array of input and output devices for receiving or sending values. Such devices include other computers, keyboards, mice, visual displays, printers, industrial equipment, and systems or machinery of all types and sizes. For example, a computer can control a network or network interface to perform various network communications upon request. The network interface may be part of the computer, or characterized as separate and remote from the computer.

A computer may be a single, physical, computing device such as a desktop computer, a laptop computer, or may be composed of multiple devices of the same type such as a group of servers operating as one device in a networked cluster, or a heterogeneous combination of different computing devices operating as one computer and linked together by a communication network. The communication network connected to the computer may also be connected to a wider network such as the internet. Thus a computer may include one or more physical processors or other computing devices or circuitry, and may also include any suitable type of memory.

A computer may also be a virtual computing platform having an unknown or fluctuating number of physical processors and memories or memory devices. A computer may thus be physically located in one geographical location or physically spread across several widely scattered locations with multiple processors linked together by a communication network to operate as a single computer.

The concept of“computer” and “processor” within a computer or computing device also encompasses any such processor or computing device serving to make calculations or comparisons as part of the disclosed system. Processing operations related to threshold comparisons, rules comparisons, calculations, and the like occurring in a computer may occur, for example, on separate servers, the same server with separate processors, or on a virtual computing environment having an unknown number of physical processors as described above.

A computer may be optionally coupled to one or more visual displays and/or may include an integrated visual display. Likewise, displays may be of the same type, or a heterogeneous combination of different visual devices. A computer may also include one or more operator input devices such as a keyboard, mouse, touch screen, laser or infrared pointing device, or gyroscopic pointing device to name just a few representative examples. Also, besides a display, one or more other output devices may be included such as a printer, plotter, industrial manufacturing machine, 3D printer, and the like. As such, various display, input and output device arrangements are possible.

Multiple computers or computing devices may be configured to communicate with one another or with other devices over wired or wireless communication links to form a network. Network communications may pass through various computers operating as network appliances such as switches, routers, firewalls or other network devices or interfaces before passing over other larger computer networks such as the internet. Communications can also be passed over the network as wireless data transmissions carried over electromagnetic waves through transmission lines or free space. Such communications include using WiFi or other Wireless Local Area Network (WLAN) or a cellular transmitter/receiver to transfer data.

“Data” generally refers to one or more values of qualitative or quantitative variables that are usually the result of measurements. Data may be considered “atomic” as being finite individual units of specific information. Data can also be thought of as a value or set of values that includes a frame of reference indicating some meaning associated with the values. For example, the number “2” alone is a symbol that absent some context is meaningless. The number “2” may be considered “data” when it is understood to indicate, for example, the number of items produced in an hour.

Data may be organized and represented in a structured format. Examples include a tabular representation using rows and columns, a tree representation with a set of nodes considered to have a parent-children relationship, or a graph representation as a set of connected nodes to name a few.

The term “data” can refer to unprocessed data or “raw data” such as a collection of numbers, characters, or other symbols representing individual facts or opinions. Data may be collected by sensors in controlled or uncontrolled environments, or generated by observation, recording, or by processing of other data. The word “data” may be used in a plural or singular form. The older plural form “datum” may be used as well.

“Database” also referred to as a “data store”, “data repository”, or “knowledge base” generally refers to an organized collection of data. The data is typically organized to model aspects of the real world in a way that supports processes obtaining information about the world from the data. Access to the data is generally provided by a “Database Management System” (DBMS) consisting of an individual computer software program or organized set of software programs that allow user to interact with one or more databases providing access to data stored in the database (although user access restrictions may be put in place to limit access to some portion of the data). The DBMS provides various functions that allow entry, storage and retrieval of large quantities of information as well as ways to manage how that information is organized. A database is not generally portable across different DBMSs, but different DBMSs can interoperate by using standardized protocols and languages such as Structured Query Language (SQL), Open Database Connectivity (ODBC), Java Database Connectivity (JDBC), or Extensible Markup Language (XML) to allow a single application to work with more than one DBMS.

Databases and their corresponding database management systems are often classified according to a particular database model they support. Examples include a DBMS that relies on the “relational model” for storing data, usually referred to as Relational Database Management Systems (RDBMS). Such systems commonly use some variation of SQL to perform functions which include querying, formatting, administering, and updating an RDBMS. Other examples of database models include the “object” model, the “object-relational” model, the“file”, “indexed file” or “flat-file” models, the “hierarchical” model, the “network” model, the “document” model, the “XML” model using some variation of XML, the “entity-attribute-value” model, and others.

Examples of commercially available database management systems include PostgreSQL provided by the PostgreSQL Global Development Group; Microsoft SQL Server provided by the Microsoft Corporation of Redmond, Washington, USA; MySQL and various versions of the Oracle DBMS, often referred to as simply “Oracle” both separately offered by the Oracle Corporation of Redwood City, California, USA; the DBMS generally referred to as “SAP” provided by SAP SE of Walldorf, Germany; and the DB2 DBMS provided by the International Business Machines Corporation (IBM) of Armonk, New York, USA.

The database and the DBMS software may also be referred to collectively as a “database”. Similarly, the term “database” may also collectively refer to the database, the corresponding DBMS software, and a physical computer or collection of computers. Thus the term “database” may refer to the data, software for managing the data, and/or a physical computer that includes some or all of the data and/or the software for managing the data.

“Display device” generally refers to any device capable of being controlled by an electronic circuit or processor to display information in a visual or tactile. A display device may be configured as an input device taking input from a user or other system (e.g. a touch sensitive computer screen), or as an output device generating visual or tactile information, or the display device may configured to operate as both an input or output device at the same time, or at different times.

The output may be two-dimensional, three-dimensional, and/or mechanical displays and includes, but is not limited to, the following display technologies: Cathode ray tube display (CRT), Light-emitting diode display (LED), Electroluminescent display (ELD), Electronic paper, Electrophoretic Ink (E-ink), Plasma display panel (PDP), Liquid crystal display (LCD), High-Performance Addressing display (HPA), Thin-film transistor display (TFT). Organic light-emitting diode display (OLED), Surface-conduction electron-emitter display (SED), Laser TV, Carbon nanotubes, Quantum dot display, Interferometric modulator display (IMOD), Swept-volume display, Varifocal mirror display, Emissive volume display, Laser display, Holographic display, Light field displays. Volumetric display, Ticker tape, Split-flap display, Flip-disc display (or flip-dot display), Rollsign, mechanical gauges with moving needles and accompanying indicia, Tactile electronic displays (aka refreshable Braille display), Optacon displays, or any devices that either alone or in combination are configured to provide visual feedback on the status of a system, such as the “check engine” light, a “low altitude” warning light, an array of red, yellow, and green indicators configured to indicate a temperature range.

“Electromagnetic Radiation” generally refers to energy radiated by electromagnetic waves. Electromagnetic radiation is produced from other types of energy, and is converted to other types when it is destroyed. Electromagnetic radiation carries this energy as it travels moving away from its source at the speed of light (in a vacuum). Electromagnetic radiation also carries both momentum and angular momentum. These properties may all be imparted to matter with which the electromagnetic radiation interacts as it moves outwardly away from its source.

Electromagnetic radiation changes speed as it passes from one medium to another. When transitioning from one media to the next, the physical properties of the new medium can cause some or all of the radiated energy to be reflected while the remaining energy passes into the new medium. This occurs at every junction between media that electromagnetic radiation encounters as it travels.

The photon is the quantum of the electromagnetic interaction, and is the basic constituent of all forms of electromagnetic radiation. The quantum nature of light becomes more apparent at high frequencies as electromagnetic radiation behaves more like particles and less like waves as its frequency increases.

“Electromagnetic Waves” generally refers to waves having a separate electrical and a magnetic component. The electrical and magnetic components of an electromagnetic wave oscillate in phase and are always separated by a 90 degree angle. Electromagnetic waves can radiate from a source to create electromagnetic radiation capable of passing through a medium or through a vacuum. Electromagnetic waves include waves oscillating at any frequency in the electromagnetic spectrum including, but not limited to radio waves, visible and invisible light. X-rays, and gamma-rays.

“Input Device” generally refers to any device coupled to a computer that is configured to receive input and deliver the input to a processor, memory, or other part of the computer. Such input devices can include keyboards, mice, trackballs, touch sensitive pointing devices such as touchpads, or touchscreens. Input devices also include any sensor or sensor array for detecting environmental conditions such as temperature, light, noise, vibration, humidity, and the like.

“Memory” generally refers to any storage system or device configured to retain data or information. Each memory may include one or more types of solid-state electronic memory, magnetic memory, or optical memory, just to name a few. Memory may use any suitable storage technology, or combination of storage technologies, and may be volatile, nonvolatile, or a hybrid combination of volatile and nonvolatile varieties. By way of non-limiting example, each memory may include solid-state electronic Random Access Memory (RAM), Sequentially Accessible Memory (SAM) (such as the First-in, First-Out (FIFO) variety or the Last-in-First-Out (LIFO) variety), Programmable Read Only Memory (PROM), Electronically Programmable Read Only Memory (EPROM), or Electrically Erasable Programmable Read Only Memory (EEPROM).

Memory can refer to Dynamic Random Access Memory (DRAM) or any variants, including static random access memory (SRAM), Burst SRAM or Synch Burst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (REDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM).

Memory can also refer to non-volatile storage technologies such as non-volatile read access memory (NVRAM), flash memory, non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Domain Wall Memory (DWM) or “Racetrack” memory, Nano-RAM (NRAM), or Millipede memory. Other non-volatile types of memory include optical disc memory (such as a DVD or CD ROM), a magnetically encoded hard disc or hard disc platter, floppy disc, tape, or cartridge media. The concept of a “memory” includes the use of any suitable storage technology or any combination of storage technologies.

“Module” or “Engine” generally refers to a collection of computational or logic circuits implemented in hardware, or to a series of logic or computational instructions expressed in executable, object, or source code, or any combination thereof, configured to perform tasks or implement processes. A module may be implemented in software maintained in volatile memory in a computer and executed by a processor or other circuit. A module may be implemented as software stored in an erasable/programmable nonvolatile memory and executed by a processor or processors. A module may be implanted as software coded into an Application Specific Information Integrated Circuit (ASIC). A module may be a collection of digital or analog circuits configured to control a machine to generate a desired outcome.

Modules may be executed on a single computer with one or more processors, or by multiple computers with multiple processors coupled together by a network. Separate aspects, computations, or functionality performed by a module may be executed by separate processors on separate computers, by the same processor on the same computer, or by different computers at different times.

“Motion Sensor” generally refers to a device configured to convert physical movement of an object into an electrical or signal. A motion sensor may be thought of as a transducer detecting physical movement and from it producing a signal (e.g. a time varying signal) based on that movement. A motion sensor may operate by detecting changes in its position relative to other objects by emitting and/or detecting electromagnetic waves. Examples include ultrasonic, infrared, video, microwave, or other such motion detectors.

In another example, a motion sensor may operate by detecting changes in the magnitude and direction of proper acceleration caused by gravity (“g-force”). Sometimes called “accelerometers,” these motion sensors can detect changes in g-forces on an object as a vector quantity, and can be used to sense changes in orientation (e.g. when the direction of weight changes), coordinate acceleration (e.g. when it produces g-force or a change in g-force), vibration, shock, and/or falling in a resistive medium. An accelerometer may thus be used to detect changes in the position, orientation, and movement of a device.

Commercially available accelerometers include piezoelectric, piezoresistive and capacitive components. Piezoelectric accelerometers may rely on piezoceramics (e.g. lead zirconate titanate) or single crystals (e.g. quartz, tourmaline). Piezoresistive accelerometers may be preferred in high shock applications. Capacitive accelerometers may use a silicon micro-machined sensing element.

A motion sensor may include multiple accelerometers. Some accelerometers are designed to be sensitive only in one direction. A motion sensor sensitive to movement in more than one direction may be constructed by integrating two accelerometers perpendicular to one another within a single package. By adding a third device oriented in a plan orthogonal to two other axes, three axes can be measured.

“Multiple” as used herein is synonymous with the term “plurality” and refers to more than one, or by extension, two or more.

“Network” or “Computer Network” generally refers to a telecommunications network that allows computers to exchange data. Computers can pass data to each other along data connections by transforming data into a collection of datagrams or packets. The connections between computers and the network may be established using either cables, optical fibers, or via electromagnetic transmissions such as for wireless network devices.

Computers coupled to a network may be referred to as “nodes” or as “hosts” and may originate, broadcast, route, or accept data from the network. Nodes can include any computing device such as personal computers, phones, servers as well as specialized computers that operate to maintain the flow of data across the network, referred to as “network devices”. Two nodes can be considered “networked together” when one device is able to exchange information with another device, whether or not they have a direct connection to each other.

Examples of wired network connections may include Digital Subscriber Lines (DSL), coaxial cable lines, or optical fiber lines. The wireless connections may include BLUETOOTH, Worldwide Interoperability for Microwave Access (WiMAX), infrared channel or satellite band, or any wireless local area network (Wi-Fi) such as those implemented using the Institute of Electrical and Electronics Engineers' (IEEE) 802.11 standards (e.g. 802.11(a), 802.11(b), 802.11(g), or 802.11(n) to name a few). Wireless links may also include or use any cellular network standards used to communicate among mobile devices including 1G, 2G, 3G, or 4G. The network standards may qualify as 1G, 2G, etc. by fulfilling a specification or standards such as the specifications maintained by International Telecommunication Union (ITU). For example, a network may be referred to as a “3G network” if it meets the criteria in the International Mobile Telecommunications-2000) (IMT-2000) specification regardless of what it may otherwise be referred to. A network may be referred to as a “4G network” if it meets the requirements of the International Mobile Telecommunications Advanced (IMTAdvanced) specification. Examples of cellular network or other wireless standards include AMPS. GSM, GPRS, UMTS, LTE, LTE Advanced. Mobile WiMAX, and WiMAX-Advanced.

Cellular network standards may use various channel access methods such as FDMA, TDMA, CDMA, or SDMA. Different types of data may be transmitted via different links and standards, or the same types of data may be transmitted via different links and standards.

The geographical scope of the network may vary widely. Examples include a body area network (BAN), a personal area network (PAN), a low power wireless Personal Area Network using IPv6 (6LoWPAN), a local-area network (LAN), a metropolitan area network (MAN), a wide area network (WAN), or the Internet.

A network may have any suitable network topology defining the number and use of the network connections. The network topology may be of any suitable form and may include point-to-point, bus, star, ring, mesh, or tree. A network may be an overlay network which is virtual and is configured as one or more layers that use or “lay on top of” other networks.

A network may utilize different communication protocols or messaging techniques including layers or stacks of protocols. Examples include the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including. e.g., IPv6), or the link layer.

“Output Device” generally refers to any device or collection of devices that is controlled by computer to produce an output. This includes any system, apparatus, or equipment receiving signals from a computer to control the device to generate or create some type of output. Examples of output devices include, but are not limited to, screens or monitors displaying graphical output, any projector a projecting device projecting a two-dimensional or three-dimensional image, any kind of printer, plotter, or similar device producing either two-dimensional or three-dimensional representations of the output fixed in any tangible medium (e.g. a laser printer printing on paper, a lathe controlled to machine a piece of metal, or a three-dimensional printer producing an object). An output device may also produce intangible output such as, for example, data stored in a database, or electromagnetic energy transmitted through a medium or through free space such as audio produced by a speaker controlled by the computer, radio signals transmitted through free space, or pulses of light passing through a fiber-optic cable.

“Pad” or “patch” generally refers to a thin flat mat or cushion. Examples include a guard worn to shield body parts against abrasion or impact, or to absorb liquids or other viscous materials. Any suitable material may be used in this context as a pad such as plastic, cloth, paper, thin metals, and the like.

As an element in a circuit, a pad or patch generally refers to a small area of electrically conductive material that may be electrically and/or physically connected to a circuit. A pad may allow for physical as well as electrical connection to the circuit such as by allowing a component or trace to be soldered to a Printed Circuit Board (PCB). A patch may refer to a pad that is attached to or incorporated into a fabric such as by weaving conductive threads into a specific predetermined area of the fabric. Pads or patches may be configured as a “surface mount” type with circuits connecting to the pad on the same surface of the board or fabric as the pad, or a “through-hole” type where pins of the components pass through the pad from one side to the other and are soldered, clamped in place, or otherwise maintained in position relative to the pad.

“Piezoresistive Effect” generally refers to an effect caused in Piezoresistive materials where the electrical resistance of the material increases as it is deformed. Examples of Piezoresistive materials include Monocrystalline Silicon, Polysilicon Thin Film, Bonded Metal Foil, Thick Film, and Sputtered Thin Film. Generally, the strain gauges are connected to form a Wheatstone bridge circuit to maximize the output of the sensor and to reduce sensitivity to errors. This is the most commonly employed sensing technology for general purpose pressure measurement.

“Pin” generally refers to a thin piece of material often having a sharpened point at one end for penetrating into and fastening to another material. A pin may have a head opposite the point that is blunt such as in the case of a nail, knitting needle, or sewing pin, or the opposing end of the pin may be attached to another item such as in the case of a pin for an electronic connector mounted to a Printed Circuit Board (PCB). A pin may be made of any suitable material such as copper, aluminum, steel, plastic, wood, and the like. Pins are often elongate structures with circular, ovular, rectangular, triangular, or any other suitable cross section. Examples of pins include nails, staples, tacks, bolts, pegs, rivets, screws, safety pins, sewing needles, or pins in an electronic connector, and the like.

“Personal computing device” generally refers to a computing device configured for use by individual people. Examples include mobile devices such as Personal Digital Assistants (PDAs), tablet computers, wearable computers installed in items worn on the human body such as in eye glasses, watches, laptop computers, portable music-video players, computers in automobiles, or cellular telephones such as smart phones. Personal computing devices can be devices that are typically not mobile such as desk top computers, game consoles, or server computers. Personal computing devices may include any suitable input/output devices and may be configured to access a network such as through a wireless or wired connection, and/or via other network hardware.

“Pressure Sensor” generally refers to a transducer configured to sense or detect a pressure local to the sensor. Types of pressure sensors include, but are not limited to, sensors that measure absolute pressure, gauge pressure, vacuum pressure, or differences between two pressures connected to each side of the sensor (differential sensor).

Any suitable pressure sensing technology may be used including, but not limited to, force collecting sensors which use a diaphragm, piston, bourdon tube, or bellows to measure strain or deflection due to applied force over an area. Examples of the force collector sensor include a Piezoresistive strain gauge which uses the piezoresistive effect of bonded or formed strain gauges to detect strain due to applied pressure. Capacitive pressure sensors use a diaphragm and pressure cavity to create a variable capacitor to detect strain due to applied pressure, capacitance decreasing as pressure deforms the diaphragm. Common technologies use metal, ceramic, and silicon diaphragms. Electromagnetic pressure sensors measure the displacement of a diaphragm by measuring changes in inductance (reluctance), measuring changes in a position as measured by a Linear Variable Differential Transformer (LVDT), measuring changes in a Hall Effect, or by measuring changes in electrical current caused by eddy currents to name a few examples. Piezoelectric pressure sensors use the piezoelectric effect in certain materials such as quartz to measure the strain upon the sensing mechanism due to pressure. Optical pressure sensors include those that use the physical change of an optical fiber to detect strain due to applied pressure. Some examples of this type utilize Fiber Bragg Gratings. Another analogous technique utilizes an elastic film constructed in layers that can change reflected wavelengths according to the applied pressure. Potentiometric pressure sensors use the motion of a wiper along a resistive mechanism to detect the strain caused by applied pressure.

Other types of pressure sensors may use other properties (such as density) to infer pressure of a gas, or liquid. For example some pressure sensors may use the changes in a resonant frequency in a sensing mechanism to measure stress, or changes in gas density, caused by applied pressure. This technology may be used in conjunction with other types of sensors such as force collectors discussed above. Alternatively, resonant technology may be employed by exposing the resonating element itself to the media, whereby the resonant frequency is dependent upon the density of the media. Sensors have been made out of vibrating wire, vibrating cylinders, quartz, and silicon MEMS. In another example, pressure sensors such as the Pirani gauge may use changes in thermal conductivity of a gas due to density changes to measure pressure. The pressure sensor such as a Hot And Cold Cathode gauge may also measure the flow of charged gas particles (ions) which varies due to density changes to measure pressure.

“Processor” generally refers to one or more electronic components configured to operate as a single unit configured or programmed to process input to generate an output. Alternatively, when of a multi-component form, a processor may have one or more components located remotely relative to the others. One or more components of each processor may be of the electronic variety defining digital circuitry, analog circuitry, or both. In one example, each processor is of a conventional, integrated circuit microprocessor arrangement, such as one or more PENTIUM, i3, i5 or i7 processors supplied by INTEL Corporation of Santa Clara, California. USA. Other examples of commercially available processors include but are not limited to the X8 and Freescale Coldfire processors made by Motorola Corporation of Schaumburg, Illinois, USA; the ARM processor and TEGRA System on a Chip (SoC) processors manufactured by Nvidia of Santa Clara, California. USA; the POWER7 processor manufactured by International Business Machines of White Plains, New York, USA; any of the FX, Phenom, Athlon, Sempron, or Opteron processors manufactured by Advanced Micro Devices of Sunnyvale, California, USA; or the Snapdragon SoC processors manufactured by Qalcomm of San Diego, California, USA.

A processor also includes Application-Specific Integrated Circuit (ASIC). An ASIC is an Integrated Circuit (IC) customized to perform a specific series of logical operations is controlling a computer to perform specific tasks or functions. An ASIC is an example of a processor for a special purpose computer, rather than a processor configured for general-purpose use. An application-specific integrated circuit generally is not reprogrammable to perform other functions and may be programmed once when it is manufactured.

In another example, a processor may be of the “field programmable” type. Such processors may be programmed multiple times “in the field” to perform various specialized or general functions after they are manufactured. A field-programmable processor may include a Field-Programmable Gate Array (FPGA) in an integrated circuit in the processor. FPGA may be programmed to perform a specific series of instructions which may be retained in nonvolatile memory cells in the FPGA. The FPGA may be configured by a customer or a designer using a hardware description language (HDL). In FPGA may be reprogrammed using another computer to reconfigure the FPGA to implement a new set of commands or operating instructions. Such an operation may be executed in any suitable means such as by a firmware upgrade to the processor circuitry.

Just as the concept of a computer is not limited to a single physical device in a single location, so also the concept of a “processor” is not limited to a single physical logic circuit or package of circuits but includes one or more such circuits or circuit packages possibly contained within or across multiple computers in numerous physical locations. In a virtual computing environment, an unknown number of physical processors may be actively processing data, the unknown number may automatically change over time as well.

The concept of a “processor” includes a device configured or programmed to make threshold comparisons, rules comparisons, calculations, or perform logical operations applying a rule to data yielding a logical result (e.g. “true” or “false”). Processing activities may occur in multiple single processors on separate servers, on multiple processors in a single server with separate processors, or on multiple processors physically remote from one another in separate computing devices.

“Proximity Sensor” generally refers to a sensor configured to generate a signal based on distance to a nearby object, or “target”, generally without requiring physical contact. Lack of mechanical physical contact between the sensor and the sensed object provides the opportunity for extra reliability and long functional life.

A proximity sensor may emit an electromagnetic field or a beam of electromagnetic radiation (e.g. infrared light, for instance), and the sensor may determine proximity based on changes in the field or return signal. The object being sensed is often referred to as the “target” or “sensor target”. Different proximity targets demand different sensors. For example, a capacitive or photoelectric sensor might be suitable for a plastic target; an inductive proximity sensor may require a metallic target.

The maximum distance that a proximity sensor can detect the target is defined as the sensor's “nominal range”. A sensor may begin to emit a signal, or may change the signal already emitted when the distance from the target to the sensor exceeds the nominal range. Some sensors allow for adjustments to the nominal range, or may be configured to return an analog or digital time varying signal based on changes on the distance to the target in time.

“Receive” generally refers to accepting something transferred, communicated, conveyed, relayed, dispatched, or forwarded. The concept may or may not include the act of listening or waiting for something to arrive from a transmitting entity. For example, a transmission may be received without knowledge as to who or what transmitted it. Likewise the transmission may be sent with or without knowledge of who or what is receiving it. To “receive” may include, but is not limited to, the act of capturing or obtaining electromagnetic energy at any suitable frequency in the electromagnetic spectrum. Receiving may occur by sensing electromagnetic radiation. Sensing electromagnetic radiation may involve detecting energy waves moving through or from a medium such as a wire or optical fiber. Receiving includes receiving digital signals which may define various types of analog or binary data such as signals, datagrams, packets and the like.

“Receiver” generally refers to a device configured to receive, for example, digital or analog signals carrying information via electromagnetic energy. A receiver using electromagnetic energy may operate with an antenna or antenna system to intercept electromagnetic waves passing through a medium such as air, a conductor such as a metallic cable, or through glass fibers. A receiver can be a separate piece of electronic equipment, or an electrical circuit within another electronic device. A receiver and a transmitter combined in one unit are called a “transceiver”.

A receiver may use electronic circuits configured to filter or separate one or more desired radio frequency signals from all the other signals received by the antenna, an electronic amplifier to increase the power of the signal for further processing, and circuits configured to demodulate the information received.

Examples of the information received include sound (an audio signal), images (a video signal) or data (a digital signal). Devices that contain radio receivers include television sets, radar equipment, two-way radios, cell phones and other cellular devices, wireless computer networks, GPS navigation devices, radio telescopes, Bluetooth enabled devices, garage door openers, and/or baby monitors.

“Rule” generally refers to a conditional statement with at least two outcomes. A rule may be compared to available data which can yield a positive result (all aspects of the conditional statement of the rule are satisfied by the data), or a negative result (at least one aspect of the conditional statement of the rule is not satisfied by the data). One example of a rule is shown below as pseudo code of an “if/then/else” statement that may be coded in a programming language and executed by a processor in a computer:

```
if(clouds.areGrey( ) and
(clouds.numberOfClouds > 100)) then {
  prepare for rain;
} else {
  Prepare for sunshine;
}
```

“Sagittal Plane” generally refers to a longitudinal anatomical plane which divides the body into right and left parts. The plane may be in the center of the body and split it into two halves (mid-sagittal) or away from the midline and split it into unequal parts (para-sagittal).

“Sensor” generally refers to a transducer configured to sense or detect a characteristic of the environment local to the sensor. For example, sensors may be constructed to detect events or changes in quantities or sensed parameters providing a corresponding output, generally as an electrical or electromagnetic signal. A sensor's sensitivity indicates how much the sensor's output changes when the input quantity being measured changes.

“Sense parameter” generally refers to a property of the environment detectable by a sensor. As used herein, sense parameter can be synonymous with an operating condition, environmental factor, sensor parameter, or environmental condition. Sense parameters may include temperature, air pressure, speed, acceleration, the presence or intensity of sound or light or other electromagnetic phenomenon, the strength and/or orientation of a magnetic or electrical field, and the like.

“Short Message Service (SMS)” generally refers to a text messaging service component of phone, Web, or mobile communication systems. It uses standardized communications protocols to allow fixed line or mobile phone devices to exchange short text messages. Transmission of short messages between a Short Message Service Center (SMSC) and personal computing device is done whenever using the Mobile Application Part (MAP) of the SS7 protocol. Messages payloads may be limited by the constraints of the signaling protocol to precisely 140 octets (140 octets*8 bits/octet=1120 bits). Short messages can be encoded using a variety of alphabets: the default GSM 7-bit alphabet, the 8-bit data alphabet, and the 16-bit UCS-2 alphabet. Depending on which alphabet the subscriber has configured in the handset, this leads to the maximum individual short message sizes of 160 7-bit characters, 140 8-bit characters, or 70 16-bit characters.

“Trace” or “track” generally refers to a conductive pathway in an electrical circuit that allows electricity to flow from one electronic device to another. Examples include lines of conductive material in a Printed Circuit Board (PCB) interconnecting components mounted to the PCB such as processors, memory, diodes, resistors, LEDs. and the like. Traces may include any suitable conductive material such as aluminum, or copper. Traces may be microscopic in size such as in the case of a microchip. Micro-sized traces used in this context are sometimes referred to as “tracks.”

“Transmit” generally refers to causing something to be transferred, communicated, conveyed, relayed, dispatched, or forwarded. The concept may or may not include the act of conveying something from a transmitting entity to a receiving entity. For example, a transmission may be received without knowledge as to who or what transmitted it. Likewise the transmission may be sent with or without knowledge of who or what is receiving it. To “transmit” may include, but is not limited to, the act of sending or broadcasting electromagnetic energy at any suitable frequency in the electromagnetic spectrum. Transmissions may include digital signals which may define various types of binary data such as datagrams, packets and the like. A transmission may also include analog signals.

Information such as a signal provided to the transmitter may be encoded or modulated by the transmitter using various digital or analog circuits. The information may then be transmitted. Examples of such information include sound (an audio signal), images (a video signal) or data (a digital signal). Devices that contain radio transmitters include radar equipment, two-way radios, cell phones and other cellular devices, wireless computer networks and network devices. GPS navigation devices, radio telescopes, Radio Frequency Identification (RFID) chips, Bluetooth enabled devices, and garage door openers.

“Transmitter” generally refers to a device configured to transmit, for example, digital or analog signals carrying information via electromagnetic energy. A transmitter using electromagnetic energy may operate with an antenna or antenna system to produce electromagnetic waves passing through a medium such as air, a conductor such as a metallic cable, or through glass fibers. A transmitter can be a separate piece of electronic equipment, or an electrical circuit within another electronic device. A transmitter and a receiver combined in one unit are called a “transceiver”.

“Triggering a Rule” generally refers to an outcome that follows when all elements of a conditional statement expressed in a rule are satisfied. In this context, a conditional statement may result in either a positive result (all conditions of the rule are satisfied by the data), or a negative result (at least one of the conditions of the rule is not satisfied by the data) when compared to available data. The conditions expressed in the rule are triggered if all conditions are met causing program execution to proceed along a different path than if the rule is not triggered.

The invention claimed is:

30. A system comprising:
a sock that includes one or more pressure sensors on a sole area of the sock, wherein the pressure sensors are configured to detect pressure caused by a person wearing the sock;
a monitoring device responsive to the one or more sensors, wherein the monitoring device is configured to receive pressure input from the pressure sensors;
wherein the monitoring device is configured to process input from the sensors to determine a measured gait for the person wearing the sock; and
wherein the monitoring device includes control logic arranged and configured to use the input from the sensors to calculate a mobility age score that is a single scalar value indicating the approximate age of the person wearing the sock based on the measured gait.

31. The system of claim 30, wherein the monitoring device is configured to determine one or more mobility metrics, and wherein the mobility metrics are optionally included in the determination of the mobility age score.

32. The system of claim 30, wherein the pressure sensors are woven into fabric of the sock.

33. The system of claim 32, wherein the pressure sensors include piezoresistive fibers that are configured to change resistance according to the pressure applied to the fabric of the sock.

34. The system of claim 30, wherein the monitoring device is configured to determine pressure gradients across the sole area of the sock.

35. The system of claim 30, wherein the sock includes 8 or more pressure sensors woven into fabric of the sock, and wherein at least one of the 8 or more pressure sensors is arranged and configured to rest adjacent to big toe, heal, and/or ball regions of the sock.

36. The system of claim 30, wherein the sock includes 25 or more pressure sensors woven into fabric of the sock.

37. The system of claim 30, wherein the one or more pressure sensors are evenly distributed across the sole area of the sock.

38. The system of claim 30, wherein pressure sensors of the one or more pressure sensors are located adjacent the ball of the foot, adjacent the midfoot region, adjacent the big toe, or any combination thereof.

39. The system of claim 30, wherein the monitoring device is configured to determine one or more of the number of steps taken, distance traveled, weight distribution and heel/toe strikes, or any combination thereof.

40. The system of claim 30, wherein the monitoring device is configured to determine stride length, cadence, and gait speed when determining the mobility age score.

41. The system of claim 30, wherein the monitoring device is configured to determine a toe off condition, and/or a heal strike when determining the mobility age score.

42. The system of claim 30, wherein the monitoring device is configured to report mobility metrics and/or the mobility age score to a remote computing device via a computer network.

43. The system of claim 30, wherein the monitoring device is configured to cross reference data received from the sensors to time of day and activities typically performed at that time of day.

44. The system of claim 30, wherein the monitoring device is configured to create and update a profile specific to the person wearing the sock based on collected data values obtained from the one or more pressure sensors.

45. The system of claim 30, wherein the monitoring device includes an accelerometer and/or a gyroscope, or any combination thereof.

46. The system of claim 45, wherein the accelerometer and/or the gyroscope are configured to provide angle inputs, and wherein the angle inputs are used by the monitoring device to determine if the person is walking in a straight line, uphill, downhill, turning, or any combination thereof.
